(19) United States
(12) Patent Application Publication
(10) Pub. No.: US 2006/0240564 A1
(43) Pub. Date: Oct. 26, 2006

(54) pH gradient ion exchange LC-MS and mass compatible buffers

(76) Inventors: Chia-Hui Shieh, Fremont, CA (US); Rong Zeng, Shanghai (CN)

Correspondence Address: Junrui Yang, 6815 Devon Way, San Jose, CA, 95129, US

(21) Appl. No.: 11/295,282

(22) Filed: Dec. 6, 2005

Related U.S. Application Data
(60) Provisional application No. 60/673,176, filed on Apr. 20, 2005.

(51) Int. Cl. G01N 33/00

(52) U.S. Cl. 436/161

(57) ABSTRACT

A system for pH gradient ion exchange LC-MS and methods of using such system are described. A buffer system for pH gradient LC-MS that are compatible with a mass spectrometer and methods of using such buffer system are also disclosed.

### CROSS REFERENCE TO RELATED APPLICATIONS

This application claims the benefits of the provisional application No. 60/673,176, filed Apr. 20, 2005, which is hereby incorporated in its entirety for all purposes.

### FIELD OF THE INVENTION

The present invention relates to liquid chromatography-mass spectrometer (LC-MS). It particularly relates to systems for pH gradient ion exchange LC-MS and methods of using such system. It also relates to buffer systems that are compatible with a mass spectrometer and methods of using such buffer system.

### BACKGROUND OF THE INVENTION

In a liquid chromatographic system or a total solution liquid chromatographic system, the liquid chromatography (LC) column is located between an injector and a detector to separate one or more constituents of interest from the various interferences in a sample to be analyzed and to permit detection of these constituents by the detector. A typical mass detector in a liquid chromatographic system can measure and provide an output in terms of mass per unit of volume or mass per unit of time of the sample's components. From such an output signal, a “chromatogram” can be provided. The chromatogram can then be used by an operator to accurately identify and quantitate the chemical components present in the sample.

A trend in chromatography has been to move to higher performance and miniature liquid chromatography columns. The reason for the strong recent trend toward miniaturization is that miniaturized liquid chromatography columns have extremely low solvent consumption and require drastically reduced volumes of sample for analysis, hence providing high efficiency, sensitive separations when samples are limited.

In liquid chromatography, high resolution has been obtained using narrow diameter columns packed with microparticles. A miniature microparticle packed liquid chromatography column is typically manufactured by packing a narrow diameter tube uniformly with separation media such as bonded silica particles, also referred to as packing material or stationary phase.

Materials commonly used for the preparation of miniature analytical columns include polymer, glass, metal, fused silica and its subgroups polymer-coated fused silica and polymer-clad fused silica. Representative metals typically include stainless steel and glass-lined stainless steel.

Miniature liquid chromatography columns include small bore, microbore and capillary columns. These columns typically have lengths ranging from about 5 mm to 300 mm, but in some instances they may approach lengths of up to 5000 mm. Small bore columns generally have inner diameters of about 2 mm, whereas microbore columns have diameters of approximately 1 mm. Fused silica and other capillary columns typically have inner diameters of less than 1 mm and often less than 0.1 mm. In fact, capillary columns having inner diameters of 0.075 mm have almost become standard for liquid chromatography mass spectrometry. Fused silica capillary columns can withstand high packing pressure, e.g., 9000 psi or greater.

Silica capillary packed with reverse phase material has been used in the proteomics field for the analysis of protein/peptides by HPLC-MS/MS. The method uses a high performance liquid chromatography (HPLC) system in conjunction with mass detector. Thousands of protein/peptides were separated by HPLC and then characterized by tandem mass. Peptide sequence is identified by matching the mass/mass (MS/MS) spectra with theoretical spectra. Protein is identified by matching peptide sequence with predict fragments from genomic or proteomics data base.

Ion exchange HPLC is widely used in the separation of proteins/peptides. It provides high resolution for the separation of biopolymers without denaturing protein/peptides. However, ion exchange HPLC requires salt gradient to elute sample from the stationary phase. The salt causes high spray current and interferes with mass signal. Therefore all the ion exchange HPLC requires extensive sample clean up to remove salt before performing mass analysis. This is time consuming. In addition, some sample may be lost during clean up steps.

Obviously, there are pressing needs for new methods and new systems for ion exchange LC-MS for the separation and analysis of biological and pharmaceutical samples.

### SUMMARY OF THE INVENTION

The present invention is directed to a system for pH gradient ion exchange LC-MS comprising an injector;

one or more HPLC pumps;

one or more LC columns independently selected from the group consisting of an ion exchange column, an integrated column, and a combination of one or more ion exchange columns and one or more LC columns suitable for multi dimensional LC-MS, and wherein at least one of said one or more LC columns is used for pH gradient LC-MS;

a buffer system comprising a buffer acid or base or a combination thereof, wherein said buffer acid or base has a buffer capacity within a pH range of from about 2 to about 10; and wherein said buffer acid or base is compatible with a mass spectrometer; and

a mass spectrometer.

In one embodiment, said multi dimensional LC-MS is two dimensional LC-MS.

In one embodiment, said mass spectrometer comprising an electro-spray ionization (ESI) interface or a matrix assisted laser desorption ionization (MALDI) interface.

In one embodiment, said one or more LC columns include an ion exchange column.

In another embodiment, said one or more LC columns include an integrated column.

In yet another embodiment, said one or more LC columns include a combination of one or more ion exchange columns and one or more LC columns suitable for two dimensional LC-MS.

In yet another embodiment, said pH gradient is a continuos pH gradient.

The present invention is also directed to a buffer system for pH gradient LC-MS. The buffer system comprises a buffer acid or base or a combination thereof, wherein said buffer acid or base has a buffer capacity within a pH range of from about 2 to about 10; and wherein said buffer acid or base is compatible with a mass spectrometer.

In certain embodiments of the invention, said buffer acid or base is selected from the group consisting of carbonic acid, formic acid, acetic acid, alkyl-COOH, substituted alkyl-COOH, alkenyl-COOH, substituted alkenyl-COOH, alkynyl-COOH, substituted alkynyl-COOH, HOOC(R¹)C=C(R²)COOH, HOOC(R¹)C(R²)COOH, ammonium hydroxide, ammonium bicarbonate, alkylamine, dialkylamine, trialkylamine, substituted alkylamine, substituted dialkylamine, substituted trialkylamine, pyridine, substitute pyridine, pyrazine, substituted pyrazine, pyridazine, substituted pyridazine, pyrimidine and substituted pyrimidine, wherein R¹ and R² are independently H, alkyl, or substituted alkyl.

In certain embodiments of the invention, said buffer acid or base is selected from the group consisting of carbonic acid, formic acid, acetic acid, trifluoroacetic acid, propionic acid, propargylic acid, maleic acid, malonic acid, 2-methyl malonic acid, 2,2-dimethyl malonic acid, 2-ethyl malonic acid, and 2,2,-diethyl malonic acid, ammonium hydroxide, ammonium bicarbonate, methylamine, ethylamine, trimethylamine, triethylamine pyridine, methyl substituted pyridine, pyrazine, pyridazine and pyrimidine.

In one embodiment, at least one of said buffer acid or base is carbonic acid.

In another embodiment, at least one of said buffer acid or base is malonic acid.

The present invention is further directed to method of using the buffer system described herein for pH gradient LC-MS, for pH gradient ion exchange LC-MS, or for pH gradient ion exchange LC-MS with either an ESI interface or MALDI interface.

The present invention is further directed to method of using the system for pH gradient ion exchange LC-MS described herein to separate, to analyze, or to separate and analyze mixtures.

The present invention is further directed to method of using the buffer system described herein to separate, to analyze, or to separate and analyze mixtures.

In certain embodiments, said mixtures are one or more species selected from the group consisting of proteins, peptides, small molecules, and biomarkers.

In one embodiment, said mixtures are proteins.

In one embodiment, said mixtures are peptides.

In one embodiment, said mixtures are small molecules.

In one embodiment, said mixtures are biomarkers.

### BRIEF DESCRIPTION OF THE DRAWINGS

FIGS. 1a and 1b show the separation of peptides from Bovine serum albumin (BSA) by ion exchange LC/MS/MS.

FIG. 2 shows the separation of protein mixture by ion exchange HPLC.

FIG. 3 shows MALDI-TOF spectrum of the collected protein mixture.

FIG. 4 shows the base peak chromatograms of nine pH fractions.

### DETAILED DESCRIPTION OF THE INVENTION

In present invention, in the term “LC-MS”, “LC” may represent LC or HPLC (high performance liquid chromatography). “MS” may represent Mass spectrometer or tandem mass spectrometry (including, but not limited to, MS, MS/MS, or MS/MS/MS).

In present invention, a buffer acid or base includes any one or a combination of its ionic and non-ionic forms as long as the form has the required buffer capacity. For example, malonic acid, HOOC(R¹)C(R²)COOH wherein R¹ and R² are H, can be in any one or a combination of the following three forms: ⁻OOCCH₂COO⁻, HOOCCH₂COO⁻, and HOOCCH₂COOH. Acetic acid can be in any one or a combination of the following two forms: CH₃COO⁻, and CH₃COOH. Trimethylamine can be in any one or a combination of the following two forms: (CH₃)₃N and (CH₃)₃NH⁺.

When a buffer acid or base is compatible with a mass spectrometer (mass compatible), it is stable at room temperature. However, when subject to an energy source in a mass spectrometer, the buffer acid or base either is volatile itself or decomposes to small molecules which are volatile. Therefore, it does not interfere with the mass spectra of the analytes. When a mass compatible buffer system is used for a pH gradient LC-MS, it eliminates the need for extensive column washing associated with the use of salt gradients.

A typical energy source in a mass spectrometer includes, but not limited to, heat in the case of electro-spray ionization (ESI) interface, or laser in the case of matrix assisted laser desorption ionization (MALDI) interface. For example, when a malonic acid buffer is subjected to heat or laser, malonic acid decomposes to form acetic acid and carbon dioxide, both are volatile and do not interfere with the mass spectra of the analytes. The decomposition of malonic acid and its derivatives can be described in the following scheme: 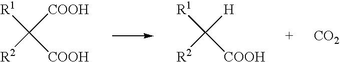
wherein R¹ and R² are independently H, alkyl, or substituted alkyl.

As used herein, “one or more” is preferably one or up to ten, one or up to six, and more preferably one or up to two.

The term “alkyl” refers to alkyl groups having from 1 to 4 carbon atoms and more preferably 1 to 3 carbon atoms. This term is exemplified by groups such as methyl, ethyl, n-propyl, iso-propyl, n-butyl, t-butyl and the like.

“Substituted alkyl” refers to an alkyl group having from 1 to 3, and preferably 1 to 2, substituents selected from the group consisting of alkoxy, acyl, acyloxy, amino, substituted amino, cyano, halogen, hydroxyl, nitro, carboxyl, and carboxyl esters.

“Alkoxy” refers to the group “alkyl-O-” which includes, by way of example, methoxy, ethoxy, n-propoxy, iso-propoxy, n-butoxy, t-butoxy, sec-butoxy and the like.

“Acyl” refers to the groups H—C(O)—, alkyl-C(O)—.

“Acyloxy” refers to alkyl-C(O)O—.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, and substituted alkynyl.

“Halo” or “halogen” refers to fluoro, chloro, bromo and iodo and preferably is fluoro or chloro.

“Carboxyl” refers to —COOH and —COO⁻.

“Carboxyl esters” refers to —C(O)O-alkyl, and —C(O)O-substituted alkyl.

“Alkenyl” refers to alkenyl group preferably having from 2 to 4 carbon atoms and more preferably 2 to 3 carbon atoms and having at least 1 site of alkenyl unsaturation. Such groups are exemplified by vinyl (ethen-1-yl), allyl and the like.

“Substituted alkenyl” refers to alkenyl groups having from 1 to 3 substituents, and preferably 1 to 2 substituents, selected from the group consisting of alkoxy, acyl, acyloxy, amino, substituted amino, cyano, halogen, hydroxyl, nitro, carboxyl, and carboxyl esters.

It is understood that the term “substituted alkenyl” includes both E (cis) and Z (trans) isomers as appropriate. The isomers can be pure isomeric compounds or mixtures of E and Z components.

“Alkynyl” refers to an unsaturated hydrocarbon having at least 1 site of alkynyl unsaturation and having from 2 to 4 carbon atoms and more preferably 2 to 3 carbon atoms. Such groups are exemplified by ethyn-1-yl, propyn-1-yl, propyn-2-yl and the like.

“Substituted alkynyl” refers to alkynyl groups having from 1 to 3 substituents, and preferably 1 to 2 substituents, selected from the group consisting of alkoxy, acyl, acyloxy, amino, substituted amino, cyano, halogen, hydroxyl, nitro, carboxyl, and carboxyl esters.

“Substituted pyridine” refers to pyridines that are substituted with from 1 to 3, preferably from 1 to 2, substituents selected from the group consisting of alkyl, substituted alkyl, alkenyl, substituted alkenyl, alkynyl, substituted alkynyl, alkoxy, acyl, acyloxy, amino, substituted amino, cyano, halogen, hydroxyl, nitro, carboxyl, and carboxyl esters.

“Substituted pyrazine” refers to pyrazines that are substituted with from 1 to 3, preferably from 1 to 2, substituents selected from the group consisting of alkyl, substituted alkyl, alkenyl, substituted alkenyl, alkynyl, substituted alkynyl, alkoxy, acyl, acyloxy, amino, substituted amino, cyano, halogen, hydroxyl, nitro, carboxyl, and carboxyl esters.

“Substituted pyridazine” refers to pyridazines that are substituted with from 1 to 3, preferably from 1 to 2, substituents selected from the group consisting of alkyl, substituted alkyl, alkenyl, substituted alkenyl, alkynyl, substituted alkynyl, alkoxy, acyl, acyloxy, amino, substituted amino, cyano, halogen, hydroxyl, nitro, carboxyl, and carboxyl esters.

“Substituted pyrimadine” refers to pyrimidines that are substituted with from 1 to 3, preferably from 1 to 2, substituents selected from the group consisting of alkyl, substituted alkyl, alkenyl, substituted alkenyl, alkynyl, substituted alkynyl, alkoxy, acyl, acyloxy, amino, substituted amino, cyano, halogen, hydroxyl, nitro, carboxyl, and carboxyl esters.

An example of pH gradient ion exchange LC-MS system comprises an injector; one or more HPLC pumps; a LC column selected from the group consisting of an ion exchange column, an integrated column, and a combination of an ion exchange column and a LC column suitable for two dimensional LC-MS; a buffer system comprising a buffer acid or base or a combination thereof, wherein said buffer acid or base has a buffer capacity within a pH range of from about 2 to about 10, and wherein said buffer acid or base is compatible with a mass spectrometer; and a mass spectrometer.

An integrated column for liquid chromatography may comprise a first column (or section) and a second column (or section). The two columns (or sections) have orthogonal separation modes. Orthogonal separation modes here mean two different separation mechanisms. When two columns have orthogonal separation modes, they are usually packed with two different stationary phases. For example, one of the columns can be selected from the group consisting of a cation exchange column, an anion exchange column, an affinity column and a metal chelating column; and the other column can be a reverse phase column. In another example, the two columns can be selected independently from the group consisting of a cation exchange column, an anion exchange column, an affinity column, a metal chelating column, and a reverse phase column.

The two columns having orthogonal separation modes can be connected through tubing and fittings; can be directly attached; or can be directly attached through nuts and fittings. An integrated column can also have two sections packed in a single column to form a mixed bed HPLC column. For example, a portion of a column is packed with strong cation exchange material and the rest of the column is packed with reverse phase material. An integrated column may further comprise one or more additional columns (or sections).

The material-used for an integrated column may be selected, but not limited to, fused silica, polymer-coated fused silica, polymer-clad fused silica, stainless steel, glass, glass-lined stainless steel, metal or polymer.

As used herein, pH gradient may include holding pH at a set value, stepwise variation of pH values, ramping pH from one value to another within certain time period (continuous pH gradient), or combinations thereof.

One embodiment of the present invention is to use pH gradient for ion exchange HPLC-MS analysis. A strong cat ion exchange column can be used for the separation. Protein/peptide sample are loaded onto column in mobile phase A. Mobile phase A contains buffer and has certain low pH value, for example, around 2.5. Under this condition all the samples are positively charged and are adsorbed by strong cat ion exchange column. The samples are then eluted by a mobile phase with increasing pH value using various pH gradients. When the pH of the mobile phase is above the pI of the species in the sample, the species lose their positive charge and elute out form the column. In this embodiment, no salt is needed for the elution. Therefore no need for sample clean up before mass analysis. This method can be used with either electro-spray ionization (ESI) interface or matrix assisted laser desorption ionization (MALDI) interface. For the ESI interface the eluent is directly sprayed into mass spectrometer without any clean up. For the MALDI interface, the eluent is collected and a small portion of the sample was put on a plate for the mass analysis.

In another embodiment, the present invention can be applied to two dimensional HPLC-MS analysis. Most two dimensional HPLC-MS require extensive column washing before reverse phase gradient and mass analysis. The pH gradient ion exchange HPLC-RPLC-MS/MS can be used without any salt clean up steps.

In certain embodiments, the buffer system described herein can be used, for example, to separate protein/peptide according to their isoelectric points (pIs). It can also be used, for example, to separate small molecules when combined with solvents suitable for reverse phase separation.

The buffer system and pH gradient ion exchange LC-MS system described herein can also be used in biomarker discovery. Biomarkers are cellular, biochemical, or molecular alterations that are measurable in biological media such as human tissues, cells, or fluids. They indicate the presence of biological events or concerted events that are directly associated with a particular disease state.

The following examples illustrate a few of numerous ways to practice present invention.

### EXAMPLES

### Example 1

### pH Gradient Ion exchange LC-MS/MS with ESI interface

This is an example of separation of peptides from Bovine serum albumin (BSA) by pH gradient ion exchange LC/MS/MS. BSA was first digested by trypsin to the peptides. The peptide mixture was separated by strong cation exchange (SCX) HPLC using pH gradients.

SCX was performed using LCQ DECAXPplus (Thermo Finnigan, San Jose, Calif.). The system was fitted with a strong cation exchange column (SCX, 320 μm, ID×100 mm, 5 μm, Column technology Inc., Calif.). The solvents were 0.05 % formic acid and 5 mM malonic acid in 20/80 acetonitrile/water. The pH value of the solvents was adjusted by aqueous ammonium (ammonium hydroxide) to yield buffer A with pH 3.0 and buffer B with pH 8.0 respectively. The peptide mixture was first loaded onto a SCX column with buffer A. The gradient started with 100% A in the first 50 min, ramped to 0% A (100% B) from 50 min to 150 min, stayed at 0% A from 150 min to 195 min and ramped to 100% A from 196 min to 210 min. The peptides were eluted out according to their pIs. The peptide eluents were directly sprayed into mass spectrometer without any pre-clean up.

The micro electro-spray interface used a 30 μm metal needle that was orthogonal to the inlet of the LCQ DecaXPplus. The mass spectrometer was set so that one full MS scan was followed by three MS/MS scans on the three most intense ions from the MS spectrum with the following Dynamic Exclusion™ settings: repeat count, 2; repeat duration, 0.5 min; exclusion duration, 3.0 min.

The acquired MS/MS spectra were automatically checked against the protein database for bovine proteins using the TurboSEQUEST program in the BioWorkS™ 3.1 cluster software suite. An accepted SEQUEST result had to have a ΔCn (The difference of Xcorr between the top hit and second top hit) score of at least 0.1 (regardless of charge state). Peptides with a +1 charge state were accepted only if they were fully tryptic and had a cross correlation (Xcorr) of at least 1.9; peptides with a +2 charge state only if they had an Xcorr>2.2; and peptides with a +3 charge state only if they had an Xcorr>3.75.

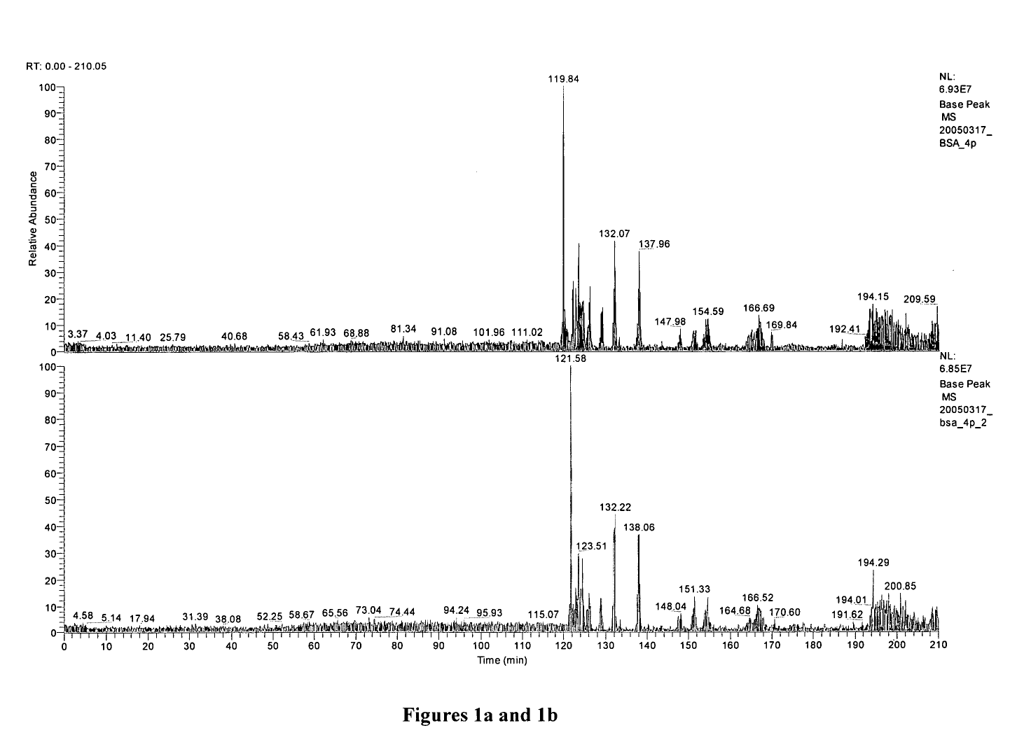

FIGS. 1a and 1b showed the separation of peptides from Bovine serum albumin (BSA) by pH gradient ion exchange LC/MS/MS.

Table 1 showed the analysis of peptides form tryptic digested BSA. Twenty-nine unique peptides were identified by ion exchange LC-MS/MS which is around 50% of total peptides. The results indicated there was no interference from buffer acids, formic acid or malonic acid. Formic acid was volatile, and Malonic acid decomposed under experimental conditions. Therefore the buffer system used in the current experiment was compatible with mass spectrometry.

TABLE 1

| Peptide Count | Unique Peptide Count | Cover Percent |
|---|---|---|
| 78 | 29 | 50.74% |

### Example 2

### pH Gradient Ion Exchange LC-MS/MS with MALDI interface

In this example the protein was isolated by pH gradient ion exchange HPLC followed by the MALDI-TOF (time of fly) analysis. A SCX column was used for the separation. The following was the condition for the pH gradient ion exchange HPLC separation:
Column: SCX 2.1×100 mm from column technology inc.,
Buffer A: 20 mM malonic acid, pH 3.0
Buffer B: 20 mM malonic acid, adjusted to pH10.0 by NH₃H₂O
Loading: 100 μg α-lactalbumin (dissolved in Buffer A)
Flow rate: 1 ml/min
Gradient: 0-100% B in 25 min

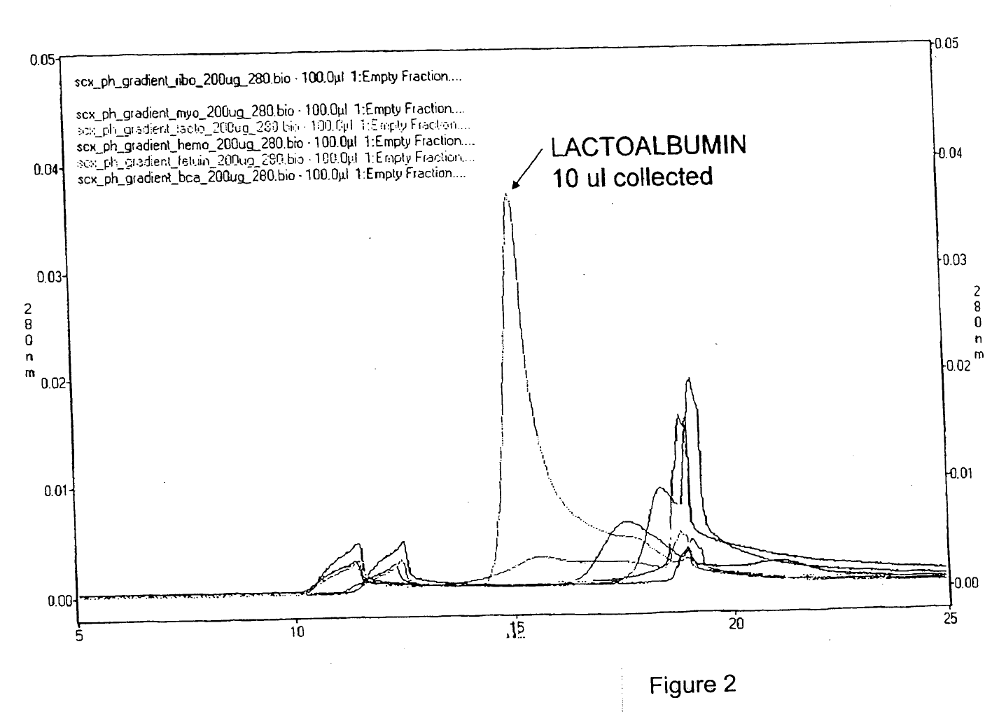

FIG. 2 showed the separation of protein mixture by pH gradient ion exchange HPLC under the conditions described above. A small fraction (10 μl ) of the sample eluent from pH gradient ion exchange HPLC was collected and used for MALDI-TOF analysis directly. No pre-sample clean up, which was required with regular salt gradient, was performed before MS/MS analysis.

All MALDI-TOF-MS experiments were performed using a Bruker Reflex III instrument. The matrix was a saturated solution of sinapic acid (SA) in solution C. Solution C was 0.1% trifluoroacetic acid in 50/50 water/acetonitrile. The sample eluent (0.5 μL) and the matrix (0.5 μl) were mixed and applied to a SCOUT 384 target and air-dried. This was followed by MALDI-TOF analysis.

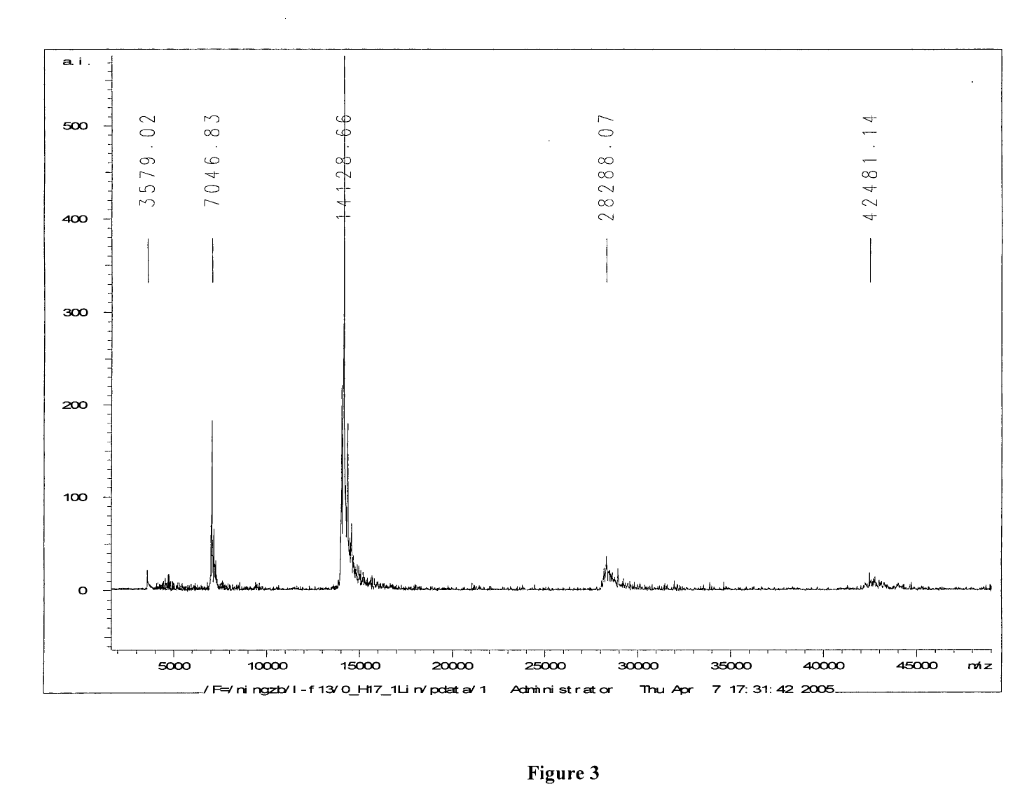

FIG. 3 showed the TOF spectrum of the sample eluent. The molecule weight around 14.1 KD was the signal corresponding to α-Lactalbumin. Formic acid evaporated, and malonic acid decomposed when laser energy was applied on the plate. Neither interfered with MS/MS analysis.

### Example 3

### Peptide identification by 2D LC/MS/MS using pH gradient and reverse phase separation

Sample Preparation

The mouse liver used in this study is C57 of 8 weeks. The C57 mouse was sacrificed and the liver was promptly removed and placed in ice-cold PBS buffer. After mincing with scissors and washing to remove blood, the liver tissue was frozen by liquid nitrogen. The frozen liver tissues were crushed in a liquid nitrogen cooled mortar, and the powder was suspended in a pre-cooled solution of 8 M Urea, 4% CHAPS, 40 mM Tris pH 8.0, 65 mM dithiothreitol (DTT) . After vortex, the lysate was stored at 4° C. for 2 hour. The lysate was sonicated at 100 W for 30 s and centrifuged at 15,000 g for 1 hour. The supernatants were then collected and the protein concentration was determined by the Bradford assay. Each 600 μg liver sample was put in 200 μL denaturing solution (6 M guanidine hydrochloride, 100 mM ammonium bicarbonate, pH 8.3); each was reduced using 2 μL of 1 M DTT. The mixture was incubated at 37° C. for 2.5 hours and then 10 μL of 1 M iodoacetamide (IAA) was added for alkylation. Afterwards the mixture was incubated for an additional 40 min at room temperature in darkness. The protein mixtures in each of the fractions were exchanged into 100 mM ammonium bicarbonate buffer, pH 8.5, with ultra-filtration through 3 kDa Microcon Centrifugal Filter Devices (Millipore, Bedford, Mass., USA). The buffer-exchanged samples were mixed together and incubated with trypsin (50:1) at 37° C. overnight. The digested peptide mixtures were lyophilized, and dissolved in 0.1% formic acid before being utilized.

2D-LC-MS/MS

Orthogonal 2D LC-MS/MS was performed using a 2D-LC-Nano-LTQ Workstation (Thermo Finnigan, San Jose, Calif., USA). The system was fitted with a strong cation exchange column (SCX, 320 μm, ID×100 mm, Column technology Inc., Fremont, Calif., USA), two C18 reversed phase trap columns (RP, 320 μm×20 mm, C18, 5 μm, Column technology Inc., Fremont, Calif., USA) and one C18 reversed phase capillary column (RP, 75 μm×150 mm, C18, 5 μm, Column technology Inc., Fremont, Calif., USA). The flow rate of the capillary column was about 200 nanoliter per min after split. The interface of LTQ is a nano-electrospray source. The mass spectrometer was set so that one full MS scan was followed by ten MS/MS scans on the ten most intense ions from the MS spectrum with the following Dynamic Exclusion™ settings: repeat count, 2; repeat duration, 0.5 min; exclusion duration, 1.5 min.

pH Gradient elution-SCX-RP-MS/MS

The SCX column was eluted by the gradient pH buffer from the sample pump. The buffer A and B of sample pump was obtained from Column Technology Inc (Fremont, Calif., USA), pH value was about 3.0 and 8.0 respectively. A total of 300 μg trypsin digested mouse liver sample was loaded to the SCX column in Buffer A (pH 3.0) by the sample pump, and the position of the 10-port valve of LTQ was set to waste.

In the first instrument method, the total acquiring time was set to 540 min. The position of the 10-port valve of LTQ was switched to source at the beginning, to waste at 180 min and back to source at 360 min. The Gradient of sample pump was ramped from 0% B to 30% B (pH from 3.0 to 3.5) at the first 180-min segment, from 30% B to 60% B (pH from 3.5 to 4.0) at the second 180-min segment and from 60% B to 70% B (pH from 4.0 to 4.5) at the third 180-min segment. The MS pump was running three 180-min RP gradient at the same time.

The second instrument method was also 540 min. The position of the 10-port valve of LTQ was switched to waste at the beginning, to source at 180 min and to waste at 360 min. The Gradient of sample pump was ramped from 70% B to 80% B (pH from 4.5 to 5.0) at the first 180-min segment, from 80% B to 85% B (pH from 5.0 to 5.5) at the second 180-min segment and from 85% B to 90% B (pH from 5.5 to 6.0) at the third 180-min segment. The RP gradient of MS pump was the same as previous.

The third instrument method was the same as the first except for the pH gradient of the sample pump, which was ramped from 90% B to 95% B (pH from 6.0 to 7.0) at the first 180-min segment, from 95% B to 100% B (pH from 7.0 to 8.0) at the second 180-min segment and from 0% B to 0% B (pH 3.0) at the third 180-min segment.

Results

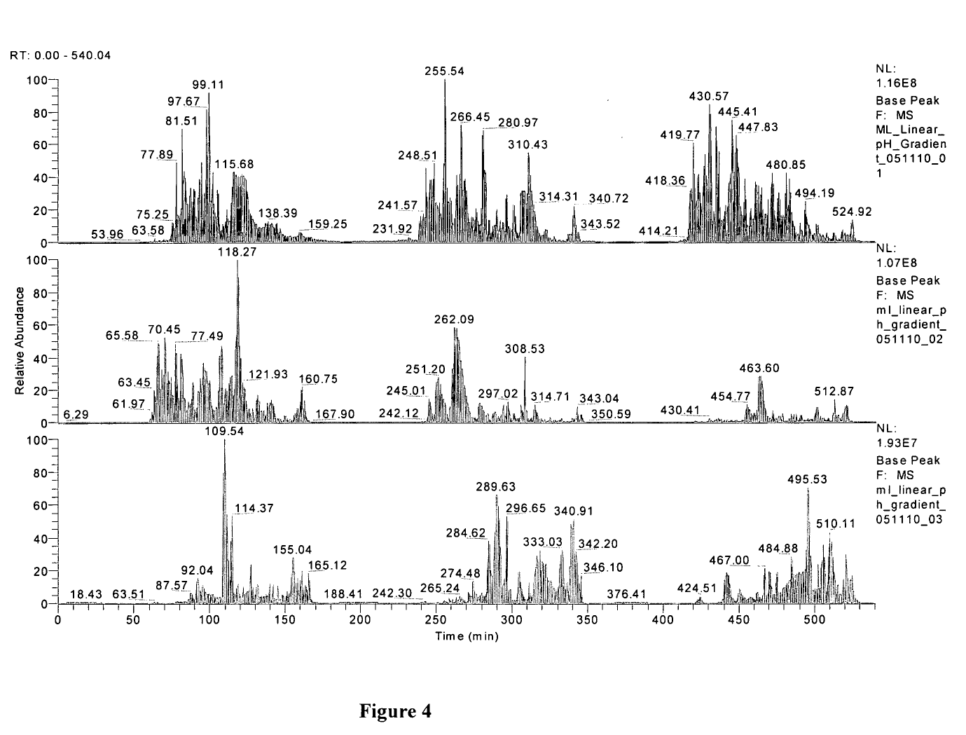

The results were summarized in FIG. 4 and Table 2. FIG. 4 shows the base peak chromatograms of nine pH fractions. A continuous pH gradient was used in the fraction of peptide mixture. Table 2 shows the peptides and proteins identified by this method. Over 4700 proteins were identified by this method.

TABLE 2

| pH | Peptide hits | peptides | Proteins |
|---|---|---|---|
| 3.0 | 1633 | 531 | 446 |
| 3.5 | 15302 | 3450 | 2125 |
| 4.0 | 15847 | 3366 | 1757 |
| 4.5 | 8985 | 2350 | 1395 |
| 5.0 | 7098 | 1657 | 1049 |
| 5.5 | 5907 | 1578 | 1014 |
| 6.0 | 4437 | 1165 | 771 |
| 7.0 | 4271 | 1146 | 766 |
| 8.0 | 2837 | 735 | 529 |
| Total | 66317 | 12057 | 4765 |

The present invention has been described with a certain degree of particularity. It is understood to those skilled in the art that the present disclosure of embodiments has been made by way of examples only and that numerous changes in the arrangement and combination of parts may be resorted without departing from the spirit and scope of the invention. While the embodiments discussed herein may appear to include some limitations as to the presentation of the information units, in terms of the format and arrangement, the invention has applicability well beyond such embodiments, which can be appreciated by those skilled in the art.

What is claimed is:

1. A system for pH gradient ion exchange LC-MS comprising
an injector;
one or more HPLC pumps;
one or more LC columns independently selected from the group consisting of an ion exchange column, an integrated column, and a combination of one or more ion exchange columns and one or more LC columns suitable for multi dimensional LC-MS, and wherein at least one of said one or more LC columns is used for pH gradient LC-MS;
a buffer system comprising a buffer acid or base or a combination thereof, wherein said buffer acid or base has a buffer capacity within a pH range of from about 2 to about 10; and wherein said buffer acid or base is compatible with a mass spectrometer; and
a mass spectrometer.

2. The system of claim 1 wherein said multi dimensional LC-MS is two dimensional LC-MS.

3. The system of claim 1 wherein said mass spectrometer comprising an ESI interface or MALDI interface.

4. The system of claim 1 wherein said one or more LC columns include an ion exchange column.

5. The system of claim 1 wherein said one or more LC columns include an integrated column.

6. The system of claim 1 wherein said one or more LC columns include a combination of one or more ion exchange columns and one or more LC columns suitable for multi dimensional LC-MS.

7. The system of claim 1 wherein said pH gradient is a continuous pH gradient.

8. The system of claim 1, wherein said buffer acid or base is selected from the group consisting of carbonic acid, formic acid, acetic acid, alkyl-COOH, substituted alkyl-COOH, alkenyl-COOH, substituted alkenyl-COOH, alkynyl-COOH, substituted alkynyl-COOH, HOOC(R¹)C=C(R²)COOH, HOOC(R¹)C(R 2)COOH, ammonium hydroxide, ammonium bicarbonate, alkylamine, dialkylamine, trialkylamine, substituted alkylamine, substituted dialkylamine, substituted trialkylamine, pyridine, substituted pyridine, pyrazine, substituted pyrazine, pyridazine, substituted pyridazine, pyrimidine and substituted pyrimidine, wherein R¹ and R² are independently H, alkyl, or substituted alkyl.

9. The system of claim 8, wherein said buffer acid or base is selected from the group consisting of carbonic acid, formic acid, acetic acid, trifluoroacetic acid, propionic acid, propargylic acid, maleic acid, malonic acid, 2-methyl malonic acid, 2,2-dimethyl malonic acid, 2-ethyl malonic acid, and 2,2,-diethyl malonic acid, ammonium hydroxide, ammonium bicarbonate, methylamine, ethylamine, trimethylamine, triethylamine, pyridine, methyl substituted pyridine, pyrazine, pyridazine and pyrimidine.

10. The system of claim 9, wherein at least one of said buffer acid or base is carbonic acid.

11. The system of claim 9, wherein at least one of said buffer acid or base is malonic acid.

12. A buffer system for pH gradient LC-MS comprising a buffer acid or base or a combination thereof, wherein said buffer acid or base has a buffer capacity within a pH range of from about 2 to about 10; and wherein said buffer acid or base is compatible with a mass spectrometer.

13. The system of claim 12 wherein said pH gradient is a continuous pH gradient.

14. The buffer system of claim 12, wherein said buffer acid or base is selected from the group consisting of carbonic acid, formic acid, acetic acid, alkyl-COOH, substituted alkyl-COOH, alkenyl-COOH, substituted alkenyl-COOH, alkynyl-COOH, substituted alkynyl-COOH, HOOC(R¹)C=C(R²)COOH, HOOC(R¹)C(R²)COOH, ammonium hydroxide, ammonium bicarbonate, alkylamine, dialkylamine, trialkylamine, substituted alkylamine, substituted dialkylamine, substituted trialkylamine, pyridine, substituted pyridine, pyrazine, substituted pyrazine, pyridazine, substituted pyridazine, pyrimidine and substituted pyrimidine, wherein R¹and R² are independently H, alkyl, or substituted alkyl.

15. The buffer system of claim 14, wherein said buffer acid or base is selected from the group consisting of carbonic acid, formic acid, acetic acid, trifluoroacetic acid, propionic acid, propargylic acid, maleic acid, malonic acid, 2-methyl malonic acid, 2,2-dimethyl malonic acid, 2-ethyl malonic acid, and 2,2,-diethyl malonic acid, ammonium hydroxide, ammonium bicarbonate, methylamine, ethylamine, trimethylamine, triethylamine, pyridine, methyl pyridine, pyrazine, pyridazine and pyrimidine.

16. The buffer system of claim 15, wherein at least one of said buffer acid or base is carbonic acid.

17. The buffer system of claim 15, wherein at least one of said buffer acid or base is malonic acid.

18. A method using any one of the buffer systems of claims 12-17 for pH gradient LC-MS.

19. A method using any one of the buffer systems of claims 12-17 for pH gradient ion exchange LC-MS.

20. A method using any one of the buffer systems of claims 12-17 for pH gradient ion exchange LC-MS with either an ESI interface or MALDI interface.

21. A method for separating, analyzing, or separating and analyzing mixtures using any one of the systems of claims 1-11.

22. A method for separating, analyzing, or separating and analyzing mixtures using any one of the buffer systems of claims 12-17.

23. The method of any one of claims 21-22, wherein said mixtures are one or more species selected from the group consisting of proteins, peptides, small molecules, and biomarkers.

24. The method of claim 23, wherein said mixtures are proteins.

25. The method of claim 23, wherein said mixtures are peptides.

26. The method of claim 23, wherein said mixtures are small molecules.

27. The method of claim 23, wherein said mixtures are biomarkers.
